(19) United States
(12) Patent Application Publication
(10) Pub. No.: US 2006/0058329 A1
(43) Pub. Date: Mar. 16, 2006

(54) Pyrazole inhibitors of the transforming growth factor

(76) Inventor: Francoise Jeanne Gellibert, Les Ulis (FR)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/522,970

(22) PCT Filed: Jul. 29, 2003
(86) PCT No.: PCT/EP03/08449

§ 371 Date: Jan. 31, 2005

(51) Int. Cl. A01N 43/42; A61K 31/44

(52) U.S. Cl. 514/279

(57) ABSTRACT

The invention relates to novel pyrazole derivatives which are inhibitors of the transforming growth factor, (“TGF”)-β signalling pathway, in particular, the phosphorylation of smad2 or smad3 by the TGF-β type I or activin-like kinase (“ALK”)-5 receptor, methods for their preparation and their use in medicine, specifically in the treatment and prevention of a disease state mediated by this pathway.

This invention relates to novel pyrazole derivatives which are inhibitors of the transforming growth factor, (“TGF”)-β signalling pathway, in particular, the phosphorylation of smad2 or smad3 by the TGF-β type I or activin-like kinase (“ALK”)-5 receptor, methods for their preparation and their use in medicine, specifically in the treatment and prevention of a disease state mediated by this pathway.

TGF-β1 is the prototypic member of a family of cytokines including the TGF-βs, activins, inhibins, bone morphogenetic proteins and Muillerian-inhibiting substance, that signal through a family of single transmembrane serine/threonine kinase receptors. These receptors can be divided into two classes, the type I or activin like kinase (ALK) receptors and type II receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The GS domain is at the amino terminal end of the intracellular kinase domain and is critical for activation by the type II receptor. Several studies have shown that TGF-β signalling requires both the ALK and type II receptors. Specifically, the type II receptor phosphorylates the GS domain of the type I receptor for TGF-β, ALK5, in the presence of TGF-β. The ALK5, in turn, phosphorylates the cytoplasmic proteins smad2 and smad3 at two carboxy terminal serines. The phosphorylated smad proteins translocate into the nucleus and activate genes that contribute to the production of extracellular matrix. Therefore, preferred compounds of this invention are selective in that they inhibit the type I receptor and thus matrix production.

Surprisingly, it has now been discovered that a class of novel pyrazole derivatives function as potent and selective non-peptide inhibitors of ALK5 kinase.

According to a first aspect, the invention provides a compound of formula (I), a pharmaceutically acceptable salt, solvate or derivative thereof; 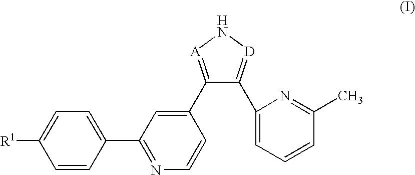
wherein
either a) A is C(R²) and D is N; or b) A is N and D is C(R²);
R¹ is selected from the list: hydrogen, C₁₋₆alkyl, C₁₋₆alkenyl, C₁₋₆alkoxy, halo, cyano, perfluoro C₁₋₆alkyl, perfluoroC₁₋₆alkoxy, —NR³R⁴, —(CH₂)ₙNR³R⁴, —O(CH₂)ₙOR⁵, —O(CH₂)ₙNR³R⁴, —O(CH₂)ₙHet, —CONR³R⁴, —CO(CH₂)ₙNR³R⁴, —SO₂R⁵, —SO₂NR³R⁴, —NR³SO₂R⁵, —NR³COR⁵, —NR³CO(CH₂)ₙNR³R⁴, Het and —O(CH₂)ₙCONR³R⁴;
R² is hydrogen or C₁₋₄alkyl;
R³ and R⁴ are independently hydrogen, C₁₋₆alkyl, Het or C₁₋₄alkoxyC₁₋₄alkyl; or R³ and R⁴ together with the nitrogen atom to which they are attached form a 3, 4, 5, 6 or 7-membered saturated or unsaturated ring which may contain one or more heteroatoms selected from N, S or O, and wherein the ring may be further substituted by one or more substituents selected from halo (such as fluoro, chloro, bromo), —CN, —CF₃, —OH, —OCF₃, C₁₋₆alkyl and C₁₋₆alkoxy;
R⁵ is hydrogen or C₁₋₆alkyl;
Het is a 5 or 6-membered C-linked heterocyclyl group which may be saturated, unsaturated or aromatic, which may contain one or more heteroatoms selected from N, S or O and which may be substituted by C₁₋₆alkyl; and
n is 1-4.

The term “C₁₋₆alkyl” as used herein, whether on its own or as part of a group, refers to a straight or branched chain saturated aliphatic hydrocarbon radical of 1 to 6 carbon atoms, unless the chain length is limited thereto, including, but not limited to methyl, ethyl, n-propyl, isopropyl, n-butyl, sec-butyl, isobutyl, tert-butyl, pentyl and hexyl.

The term “alkenyl” as a group or part of a group refers to a straight or branched chain mono- or poly-unsaturated aliphatic hydrocarbon radical containing the specified number(s) of carbon atoms. References to “alkenyl” groups include groups which may be in the E- or Z-form or mixtures thereof.

The term “alkoxy” as a group or part of a group refers to an alkyl ether radical, wherein the term “alkyl” is defined above. Such alkoxy groups in particular include methoxy, ethoxy, n-propoxy, iso-propoxy, n-butoxy, isobutoxy, sec-butoxy and tert-butoxy.

The term “perfluoroalkyl” as used herein includes compounds such as trifluoromethyl.

The term “perfluoroalkoxy” as used herein includes compounds such as trifluoromethoxy.

The terms “halo” or “halogen” are used interchangeably herein to mean radicals derived from the elements chlorine, fluorine, iodine and bromine.

The term “heterocyclyl” as used herein includes cyclic groups containing 5 to 7 ring-atoms up to 4 of which may be hetero-atoms such as nitrogen, oxygen and sulfur, and may be saturated, unsaturated or aromatic. Examples of heterocyclyl groups are furyl, thienyl, pyrrolyl, pyrrolinyl, pyrrolidinyl, imidazolyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, pyridyl, piperidinyl, dioxanyl, morpholino, dithianyl, thiomorpholino, pyridazinyl, pyrimidinyl, pyrazinyl, piperazinyl, sulfolanyl, tetrazolyl, triazinyl, azepinyl, oxazepinyl, thiazepinyl, diazepinyl and thiazolinyl. In addition, the term heterocyclyl includes fused heterocyclyl groups, for example benzimidazolyl, benzoxazolyl, imidazopyridinyl, benzoxazinyl, benzothiazinyl, oxazolopyridinyl, benzofuranyl, quinolinyl, quinazolinyl, quinoxalinyl, dihydroquinazolinyl, benzothiazolyl, phthalimido, benzofuranyl, benzodiazepinyl, indolyl and isoindolyl.

Preferably R¹ is C₁₋₆alkoxy, halo, cyano, perfluoroC₁₋₆alkoxy, —NR³R⁴, —(CH₂)ₙNR³R⁴, —O(CH₂)ₙNR³R⁴, —O(CH₂)ₙHet (where Het is preferably imidazolyl), —CONR³R⁴, —SO₂R⁵, —NR³CO(CH₂)ₙNR³R⁴, Het (preferably imidazolyl) or —O(CH₂)ₙCONR³R⁴.

When A is C(R²), R² is preferably hydrogen or C₁₋₄alkyl.

When D is C(R²), R² is preferably hydrogen.

Preferably, R³ and R⁴ are independently hydrogen, methyl, Het (preferably imidazolyl or tetrahydropyranyl) or C₁₋₄alkoxyC₁ alkyl; or R³ and R⁴ together with the atom to which they are attached form a morpholine, piperidine, pyrrolidine or piperazine ring.

It will be appreciated that the present invention is intended to include compounds having any combination of the preferred groups listed hereinbefore.

Preferably
either a) A is C(R²) and D is N; or b) A is N and D is C(R²);
R¹ is C₁₋₆alkoxy, halo, cyano, perfluoroC₁₋₆alkoxy, —NR³R⁴, —(CH₂)ₙNR³R⁴, —O(CH₂)ₙNR³R⁴, —O(CH₂)ₙHet (where Het is preferably imidazolyl), —CONR³R⁴, —SO₂R⁵, —NR³CO(CH₂)ₙNR³R⁴, Het (preferably imidazolyl) or —O(CH₂)ₙCONR³R⁴;
R² is hydrogen or methyl;
R³ and R⁴ are independently hydrogen, methyl, Het (preferably imidazolyl or tetrahydropyranyl) or C₁₋₄alkoxyC₁₋₄alkyl; or R³ and R⁴ together with the nitrogen atom to which they are attached form a morpholine, piperidine, pyrrolidine or piperazine ring, which ring may be further substituted by one or more substituents selected from halo —CN, —CF₃, —OH, —OCF₃, C₁-alkyl and C₁₋₆alkoxy (preferably C₁₋₆alkyl or C₁₋₆alkoxy);
R⁵ is hydrogen or C₁-alkyl;
Het is a 5 or 6-membered C-linked heterocyclyl group which may be saturated, unsaturated or aromatic, which may contain one or more heteroatoms selected from N, S or O and which may be substituted by C₁₋₆alkyl; and
n is 1-3.

Compounds of formula (I) which are of special interest as agents useful in the treatment or prophylaxis of disorders characterised by the overexpression of TGF-β are:
2-{4-(1-methyl-imidazol-4-yl)methyloxy]-phenyl}-4-(3-(6-methyl-pyridin-2-yl)-1H-pyrazol-4-yl)pyridine (Example 1);
2-[4-(ethylsulfonyl)phenyl]-4-[3-(6-methyl-pyridin-2-yl)-1H-pyrazol-4-yl]pyridine (Example 2);
4-[3-(6-methylpyridin-2-yl)-1H-pyrazol-4-yl]-2-[4-(pyrrolidin-1-ylmethyl)phenyl]pyridine (Example 3);
4-{4-[5-methyl-3-(6-methylpyridin-2-yl)-1H-pyrazol-4-yl]pyridin-2-yl}benzyl)morpholine (Example 7); and
3-[2-(4-(2-(pyrolidin-1-yl)ethoxy)phenyl)pyridin-4-yl]-4-[6-methylpyridin-2-yl]-1H-pyrazole (Example 22);
and pharmaceutically acceptable salts, solvates and derivatives thereof.

For the avoidance of doubt, unless otherwise indicated, the term substituted means substituted by one or more defined groups. In the case where groups may be selected from a number of alternative groups, the selected groups may be the same or different.

For the avoidance of doubt, the term independently means that where more than one substituent is selected from a number of possible substituents, those substituents may be the same or different.

As used herein the term “pharmaceutically acceptable derivative” means any pharmaceutically acceptable salt, solvate, ester or amide, or salt or solvate of such ester or amide, of the compound of formula (I), or any other compound which upon administration to the recipient is capable of providing (directly or indirectly) the a compound of formula (I) or an active metabolite or residue thereof, e.g., a prodrug. Preferred pharmaceutically acceptable derivatives according to the invention are any pharmaceutically acceptable salts, solvates or prodrugs.

Suitable pharmaceutically acceptable salts of the compounds of formula (I) include acid salts, for example sodium, potassium, calcium, magnesium and tetraalkylammonium and the like, or mono- or di-basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like. Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Hereinafter, compounds, their pharmaceutically acceptable salts, their solvates and polymorphs, defined in any aspect of the invention (except intermediate compounds in chemical processes) are referred to as “compounds of the invention”.

The compounds of the invention may exist in one or more tautomeric forms. All tautomers and mixtures thereof are included in the scope of the present invention.

Compounds of the invention may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

Compounds of the invention may be prepared, in known manner in a variety of ways. In the following reaction schemes and hereafter, unless otherwise stated R¹ to R⁵ and n are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic) etc . . . . (IVa), (IVb), (IVc) etc.

Compounds of formula (Ia), i.e. compounds of general formula (I) where A is C(R²) and D is N, may be prepared according to reaction scheme 1 from compounds of formula (II). Compounds of formula (II) are reacted with boron-containing compounds of formula (III) using Suzuki coupling conditions (see Miyaura et al. Chem. Rev. 1995, 95: 2457) to give compounds of formula (IV). Preferably reaction is carried out in the presence of a suitable base such as sodium carbonate, potassium carbonate, potassium hydroxide or sodium hydroxide, in the presence of a palladium or nickel catalyst, preferably at elevated temperature for a period of between 30 minutes and 48 hours. Preferred catalysts include tetrakis(triphenlyphosphine) palladium(0), palladium(II) acetate, dichlorobis(triphenylphosphine) palladium(II), tris(dibenzylideneacetone) dipalladium(0) and dichlorobis(triphenylphosphine) nickel. Compounds of formula (IV) may be deprotected under acidic conditions (preferably hydrochloric acid) to give compounds of formula (Ia).

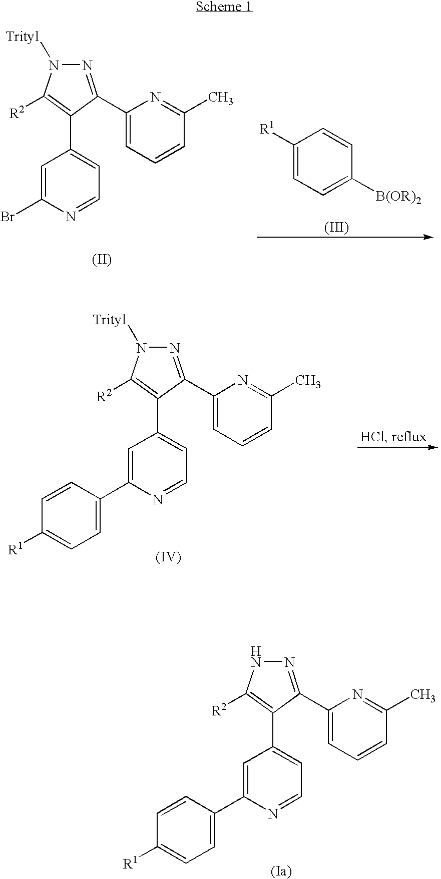

Compounds of formula (Ib), i.e. compounds of general formula (I) where A is N and D is CH, may be prepared according to reaction scheme 2 from compounds of formula (V), by reacting compounds of formula (V) with dimethylformamide dimethyl acetal and acetic acid in a solvent such as DMF at room temperature, followed by treatment with hydrazine.

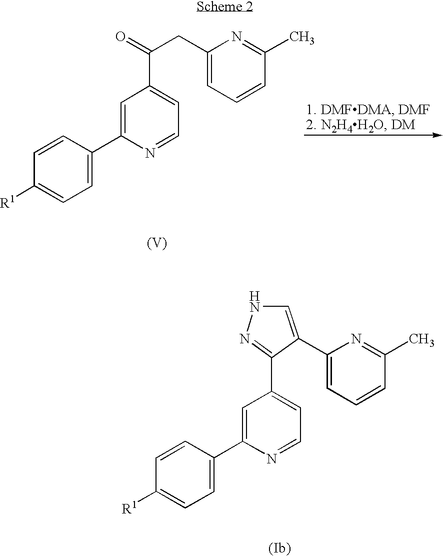

Compounds of formula (V) may be prepared using Suzuki coupling methodology (see reaction scheme 1) from compounds of formula (VI) according to reaction scheme 3. Compounds of formula (VI) may in turn be prepared in two steps from 2-bromo-4-pyridinecarboxylic acid.

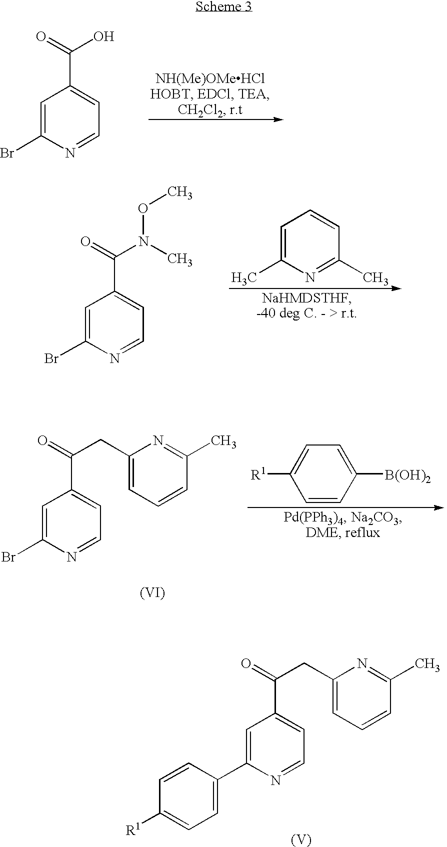

Compounds of formula (IVa), i.e. compounds of formula (IV) (see reaction scheme 1) where R¹ is OR (where R is C₁₋₆alkyl, —(CH₂)ₙOR⁵, —(CH₂)ₙNR³R⁴ or —(CH₂)ₙHet), may be prepared from compounds of formula (VIII) according to reaction scheme 4, by reaction with RX (where X is a leaving group such as halogen) in the presence of base such as potassium carbonate or sodium hydride in a solvent such as dimethylformamide.

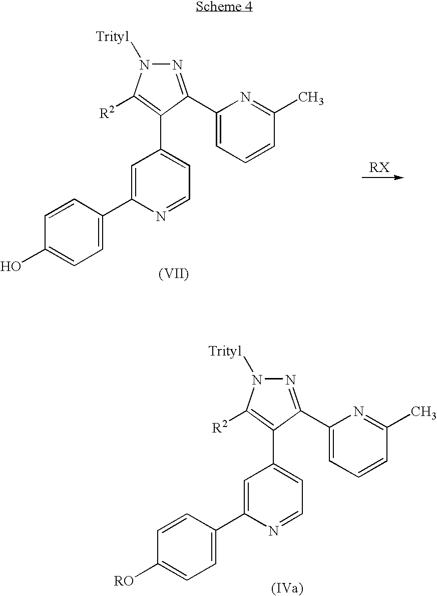

Compounds of formula (IVb), i.e. compounds of formula (IV) (see reaction scheme 1) where R¹ is CONR³R⁴, may be prepared according to reaction scheme 5, by reacting compounds of formula (VIII) with R³R⁴NH preferably in the presence of hydroxybenzotriazole and 1-[3-(dimethylamino)propyl]-3-ethylcarbodiimide hydrochloride.

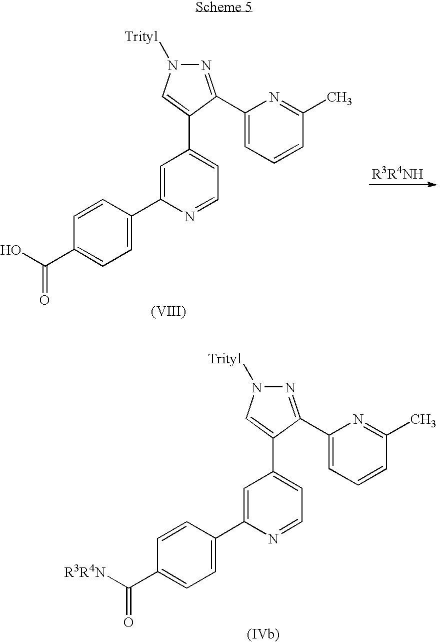

Compounds of formula (IVc), i.e. compounds of formula (IV) (see reaction scheme 1) where R¹ is —CH₂NR³R⁴, may be prepared according to reaction scheme 6 by reacting compounds of formula (IX) with R³R⁴NH in the presence of a reducing agent, preferably sodium triacetoxyborohydride in acetic acid, in a solvent such as dichloroethane at room temperature.

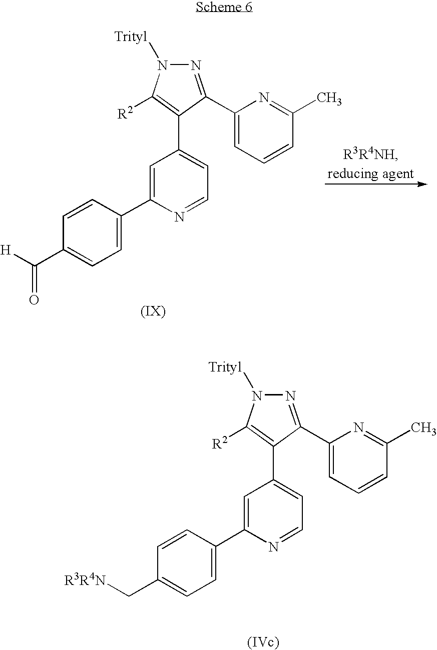

Compounds of formula (VII), (VIII) and (IX) may be prepared by Suzuki coupling of compounds of formula (II) and the appropriate boron-containing compound, using conditions analogous to those described for reaction scheme 1.

The skilled person will appreciate that compounds of formula (IVa), (IVb) and (IVc) may also be prepared directly by Suzuki coupling of compounds of formula (II) with the appropriate boron-containing compound.

Compounds of formula (II) may be prepared according to reaction scheme 7. Firstly, 2-bromo-4-methylpyridine may be coupled to compounds of formula (X) to give compounds of formula (XI). Preferred reaction conditions comprise treatment with a base such as sodium bis(trimethylsilyl)amide or potassium bis(trimethylsilyl)amide in tetrahydrofuran at a range of temperature from −70° C. to 0° C. Compounds of formula (XI) may then be reacted with dimethylformamide dimethyl acetal and acetic acid in a solvent such as DMF at room temperature followed by treatment with hydrazine to give compounds of formula (XII) where R² is hydrogen. To prepare compounds of formula (XII) where R² is methyl, N,N-dimethylacetamide acetal is used instead of dimethylformamide dimethyl acetal. Reaction of compounds of formula (XII) with trityl chloride gives compounds of formula (II).

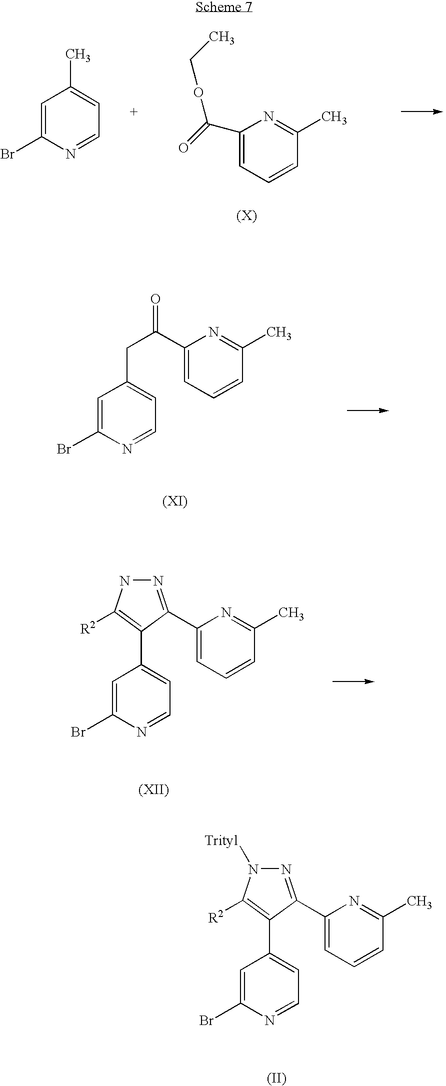

Further details for the preparation of compounds of formula (I) are found in the examples.

The compounds of the invention may be prepared singly or as compound libraries comprising at least 2, for example 5 to 1,000 compounds, and more preferably 10 to 100 compounds. Libraries of compounds of the invention may be prepared by a combinatorial ‘split and mix’ approach or by multiple parallel synthesis using either solution phase or solid phase chemistry, by procedures known to those skilled in the art. Thus according to a further aspect there is provided a compound library comprising at least 2 compounds of the invention.

Activation of the TGF-β1 axis and expansion of extracellular matrix are early and persistent contributors to the development and progression of chronic renal disease and vascular disease. Border W. A., et al., N. Engl. J. Med., 1994; 331(19), 1286-92. Further, TGF-β1 plays a role in the formation of fibronectin and plasminogen activator inhibitor-1, components of sclerotic deposits, through the action of smad3 phosphorylation by the TGF-β1 receptor ALK5. Zhang Y., et al., Nature, 1998; 394(6696), 909-13; Usui T., et al, Invest. Ophthalmol. Vis. Sci., 1998; 39(11), 1981-9. Progressive fibrosis in the kidney and cardiovascular system is a major cause of suffering and death and an important contributor to the cost of health care. TGF-β1 has been implicated in many renal fibrotic disorders. Border W. A., et al, N. Engl. J. Med., 1994; 331(19), 1286-92. TGF-β1 is elevated in acute and chronic glomerulonephritis Yoshioka K., et al, Lab. Invest., 1993; 68(2), 154-63, diabetic nephropathy Yamamoto, T., et al, 1993, PNAS 90, 1814-1818., allograft rejection, HIV nephropathy and angiotensin-induced nephropathy Border W. A., et al., N. Engl. J. Med., 1994; 331(19), 1286-92. In these diseases the levels of TGF-β1 expression coincide with the production of extracellular matrix. Three lines of evidence suggest a causal relationship between TGF-β1 and the production of matrix. First, normal glomeruli, mesangial cells and non-renal cells can be induced to produce extracellular-matrix protein and inhibit protease activity by exogenous TGF-β1 in vitro. Second, neutralizing anti-bodies against TGF-β1 can prevent the accumulation of extracellular matrix in nephritic rats. Third, TGF-β1 transgenic mice or in vivo transfection of the TGF-β1 gene into normal rat kidneys resulted in the rapid development of glomerulosclerosis. Kopp J. B., et al, Lab. Invest., 1996; 74(6), 991-1003. Thus, inhibition of TGF-β1 activity is indicated as a therapeutic intervention in chronic renal disease.

TGF-β1 and its receptors are increased in injured blood vessels and are indicated in neointima formation following balloon angioplasty Saltis J., et al, Clin. Exp. Pharmacol. Physiol., 1996; 23(3), 193-200. In addition TGF-β1 is a potent stimulator of smooth muscle cell (“SMC”) migration in vitro and migration of SMC in the arterial wall is a contributing factor in the pathogenesis of atherosclerosis and restenosis. Moreover, in multivariate analysis of the endothelial cell products against total cholesterol, TGF-β receptor ALK5 correlated with total cholesterol (P<0.001) Blann A. D., et al., Atherosclerosis, 1996; 120(1-2), 221-6. Furthermore, SMC derived from human atherosclerotic lesions have an increased ALK5/TGF-β type II receptor ratio. Because TGF-β1 is over-expressed in fibroproliferative vascular lesions, receptor-variant cells would be allowed to grow in a slow, but uncontrolled fashion, while overproducing extracellular matrix components McCaffrey T. A., et al., Jr., J. Clin. Invest., 1995; 96(6), 2667-75. TGF-β1 was immunolocalized to non-foamy macrophages in atherosclerotic lesions where active matrix synthesis occurs, suggesting that non-foamy macrophages may participate in modulating matrix gene expression in atherosclerotic remodelling via a TGF-β-dependent mechanism. Therefore, inhibiting the action of TGF-β1 on ALK5 is also indicated in atherosclerosis and restenosis.

TGF-β is also indicated in wound repair. Neutralizing antibodies to TGF-β1 have been used in a number of models to illustrate that inhibition of TGF-β1 signalling is beneficial in restoring function after injury by limiting excessive scar formation during the healing process. For example, neutralizing antibodies to TGF-β1 and TGF-β2 reduced scar formation and improved the cytoarchitecture of the neodermis by reducing the number of monocytes and macrophages as well as decreasing dermal fibronectin and collagen deposition in rats Shah M., J. Cell. Sci., 1995, 108, 985-1002. Moreover, TGF-β antibodies also improve healing of corneal wounds in rabbits Moller-Pedersen T., Curr. Eye Res., 1998, 17, 736747, and accelerate wound healing of gastric ulcers in the rat, Ernst H., Gut, 1996, 39, 172-175. These data strongly suggest that limiting the activity of TGF-β would be beneficial in many tissues and suggest that any disease with chronic elevation of TGF-β would benefit by inhibiting smad2 and smad3 signalling pathways.

TGF-β is also implicated in peritoneal adhesions Saed G. M., et al, Wound Repair Regeneration, 1999 November-December, 7(6), 504-510. Therefore, inhibitors of ALK5 would be beneficial in preventing peritoneal and sub-dermal fibrotic adhesions following surgical procedures.

TGF-β is also implicated in photoaging of the skin (see Fisher G J. Kang S W. Varani J. Bata-Csorgo Z. Wan Y S. Data S. Voorhees J J., Mechanisms of photoaging and chronological skin ageing, Archives of Dermatology, 138(11):1462-1470, 2002 November and Schwartz E. Sapadin A N. Kligman L H. “Ultraviolet B radiation increases steady state mRNA levels for cytokines and integrins in hairless mouse skin-modulation by topical tretinoin”, Archives if Dermatological Research, 290(3):137-144, 1998 March.)

Therefore according to a further aspect, the invention provides the use of a compound defined in the first aspect in the preparation of a medicament for treating or preventing a disease or condition mediated by ALK-5 inhibition.

Preferably the disease or condition mediated by ALK-5 inhibition is selected from the list: chronic renal disease, acute renal disease, wound healing, arthritis, osteoporosis, kidney disease, congestive heart failure, ulcers (including diabetic ulcers, chronic ulcers, gastric ulcers, and duodenal ulcers), ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to kidney fibrosis, lung fibrosis and liver fibrosis, for example, hepatitis B virus (HBV), hepatitis C virus (HCV), alcohol-induced hepatitis, haemochromatosis, primary biliary cirrhosis, restenosis, retroperitoneal fibrosis, mesenteric fibrosis, endometriosis, keloids, cancer, abnormal bone function, inflammatory disorders, scarring and photaging of the skin.

More preferably the disease or condition mediated by ALK-5 inhibition is fibrosis. Preferably kidney fibrosis.

It will be appreciated that references herein to treatment extend to prophylaxis as well as the treatment of established conditions.

Compounds of the invention may be administered in combination with other therapeutic agents, for example antiviral agents for liver diseases, or in combination with ACE inhibitors or angiotensin II receptor antagonists for kidney diseases.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

No toxicological effects are indicated when a compound of the invention is administered in the above-mentioned dosage range.

All publications, including, but not limited to, patents and patent applications cited in this specification, are herein incorporated by reference as if each individual publication were specifically and individually indicated to be incorporated by reference herein as though fully set forth.

It will be appreciated that the invention includes the following further aspects. The preferred embodiments described for the first aspect extend these further aspects:
i) a pharmaceutical composition comprising a compound of the invention and a pharmaceutically acceptable carrier or diluent;
ii) a compound of the invention for use as a medicament;
iii) a method of treatment or prophylaxis of a disorder selected from chronic renal disease, acute renal disease, wound healing, arthritis, osteoporosis, kidney disease, congestive heart failure, ulcers (including diabetic ulcers, chronic ulcers, gastric ulcers, and duodenal ulcers), ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to kidney fibrosis, lung fibrosis and liver fibrosis, for example, hepatitis B virus (HBV), hepatitis C virus (HCV), alcohol-induced hepatitis, haemochromatosis, primary biliary cirrhosis, restenosis, retroperitoneal fibrosis, mesenteric fibrosis, endometriosis, keloids, cancer, abnormal bone function, inflammatory disorders, scarring and photoaging of the skin, in mammals, which comprises administration to the mammal in need of such treatment, an effective amount of a compound of the invention; and
iv) a combination of a compound of the invention with an ACE inhibitor or an angiotensin II receptor antagonist.

According to a further aspect, the invention provides a compound of formula (I), a pharmaceutically acceptable salt, solvate or derivative thereof; 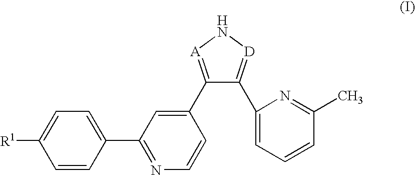
wherein
A is C(R²) and D is N;
R¹ is selected from H, C₁₋₆alkyl, C₁₋₆alkenyl, C₁₋₆alkoxy, halo, cyano, perfluoro C₁₋₆alkyl, perfluoroC₁₋₆alkoxy, —NR³R⁴, —(CH₂)ₙNR³R⁴, —O(CH₂)ₙOR⁵, —O(CH₂)ₙNR³R⁴, —CONR³R⁴, —CO(CH₂)ₙNR³R⁴, —SO₂R⁵, —SO₂NR³R⁴, —NR³SO₂R⁵ and —NR³COR⁵;
R² is selected from H, halo, C₁₋₆alkyl and —NR³R⁴;
R³, R⁴ and R⁵ are independently selected from H or C₁₋₆alkyl; or R³ and R⁴ together with the atom to which they are attached form a 3, 4, 5, 6 or 7-membered saturated or unsaturated ring which may contain one or more heteroatoms selected from N, S or O, and wherein the ring may be further substituted by one or more substituents selected from halo (such as fluoro, chloro, bromo), —CN, —CF₃, —OH, —OCF₃, C₁₋₆alkyl and C₁₋₆ alkoxy; and
n is 1-4.

The following non-limiting examples illustrate the present invention.

Abbreviations

AcOH—acetic acid
Binap—2,2′-Bis(diphenylphosphino)-1,1′-binaphthyl
CH₂Cl₂—dichloromethane
DCE—dichloroethane
DME—1,2-Dimethoxyethane
DMF—dimethylformamide
DMF.DMA—dimethylformamide dimethylacetal
EDCl—1-(3-dimethylaminopropyl)-3-ethylcarbodiimide hydrochloride
HOBT—1-hydroxybenzotriazole hydrate
NaHMDS—sodium bis(trimethylsilyl)amide
NaHBOAc₃—sodium triacetoxyborohydride
MeCN—acetonitrile
Pd₂(dba)₃—tris(dibenzylideneacetone)dipalladium(0)
Pd(PPh₃)₄—tetrakis(triphenlyphosphine) palladium(0)
SOCl₂—thionyle chloride
TEA—triethylamine
THF—tetrahydrofuran
t-BuOK—potassium tert-butoxide

### Intermediate 1: 2-[2-Bromo-pyridin-4-yl]-1-(6-methyl-pyridin-2-yl)-ethanone

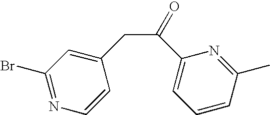

To a solution of 2-bromo-4-methyl-pyridine (5 g, 29 mmol) in dry THF (70 ml), was added dropwise a solution of sodium bis-(trimethylsilyl)amide 2M in THF (32 ml, 2.2eq) at −30° C. under nitrogen. The mixture was stirred at −30° C. for 1 h, then 6-methylpicolinic acid methyl ester (4.82 g, 32.3 mmol, 1.1 eq) was added. The reaction mixture was stirred at room temperature overnight. Diethyl ether was added and the precipitated solid filtered and washed with diethyl ether. The solid was diluted with saturated NH₄Cl solution and the aqueous phase extracted with ethyl acetate. The organic layer was dried over Na₂SO₄ and concentrated. The resulting orange powder was washed with pentane to give the title compound as a yellow solid (5.84 g, 70%); [APCI MS] m/z 292 (MH+).

### Intermediate 2: 2-Bromo-4-(3-(6-methyl-pyridin-2-yl)-1H-pyrazol-4-yl)pyridine

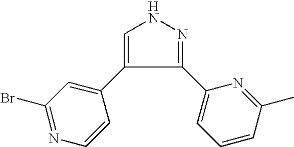

A solution of intermediate 1 (5.84 g, 20 mmol) in dry DMF (20 ml) under nitrogen was treated with glacial acetic acid (2.4eq, 2.76 ml) over 2 min. DMF.DMA (1.5eq., 4 ml) was added dropwise and the mixture stirred at room temperature under nitrogen for 1 h. Hydrazine monohydrate (7.5 eq, 91 ml, 1.876 mol) was added dropwise at room temperature and the resulting mixture heated at 50° C. for 3 h. The reaction mixture was poured into water (300 ml) and extracted with CH₂Cl₂. The organic phases were combined, dried over Na₂SO₄ and filtered. The solvent was evaporated under reduced pressure to afford a brown oil which after purification by chromatography on silica gel (eluent:CH₂Cl₂/CH₃OH 98:2) gave the title compound as a yellow solid (3.07 g, 49%); [APCI MS] m/z 315 (MH+).

### Intermediate 3: 2-bromo-4-[5-methyl-3-(6-methylpyridin-2-yl)-1H-pyrazol-4-yl]pyridine

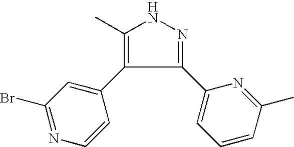

Intermediate 1 (2 g, 6.9 mmol) was reacted with N,N-dimethylacetamide dimethylacetal (1.38 g, 10 mmol) as described for intermediate 2 to afford the title compound as a brown solid (0.9 g, 39.8%); [APCI MS] m/z 328 (MH⁻).

### Intermediate 4: 2-bromo-4-[3-(6-methylpyridin-2-yl)-1-trityl-1H-pyrazol-4-yl]pyridine

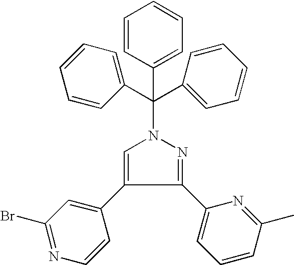

Intermediate 2 (3.07 g, 9.8 mmol) and trityl chloride (1.5 eq, 4.1 g, 14.7 mmol) were reacted with potassium carbonate (3eq, 29.4 mmol) in acetone (100 ml). The reaction mixture was subsequently heated to reflux and stirred for 24 hours. The reaction mixture was filtered, the filtrate concentrated and then partitioned between CH₂Cl₂ and H₂O. The organic phase was dried over Na₂SO₄ and concentrated. The resulting crude material was purified by flash chromatography on silica gel, eluting with CH₂Cl₂/MeOH (98:2) to give to afford the title compound as the major isomer of a mixture of the two isomers, as a light yellow solid (4.9 g, 90%); [APCI MS] m/z 558 (MH+).

### Intermediate 5: 2-bromo-4-[5-methyl-3-(6-methylpyridin-2-yl)-1-trityl-1H-pyrazol-4-yl]pyridine

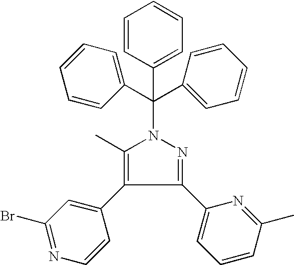

Intermediate 3 (0.9 g, 2.74 mmol) and trityl chloride (0.84 g, 3 mmol) were reacted as described for intermediate 4 to afford the title compound as a mixture of the two isomers, as a white powder (1.5 g, 95.87%); [APCI MS] m/z 329 (MH+, loss of trityl).

### Intermediate 6: 4-{4-[3-(6-Methylpyridin-2-yl)-1-trityl-1H-pyrazol-4-yl]pyridin-2-yl}-phenol

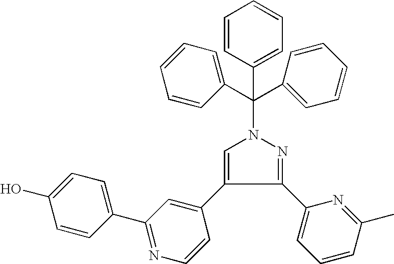

To a solution of intermediate 4 (2 g, 3.6 mmol) in a mixture of DME (36 ml) and water (18 ml) were added tetrakis triphenylphosphine palladium (0.2 g), Na₂CO₃ (0.99 g) and 4-hydroxyphenyl boronic acid pinacol ester (1.4 eq, 1.15 g, 4.32 mmol). The resulting mixture was heated under reflux overnight. The cooled mixture was poured into water and extracted with CH₂Cl₂. The organic phase was washed with water, dried over Na₂SO₄ and filtered. Evaporation of the solvent in vacuo gave a crude oil which was purified by chromatography on silica gel (CH₂Cl₂/MeOH 95:5) to give the title compound as a white solid (1.7 g, 83%), which contained the 2-trityl isomer as a minor component; [APCI MS] m/z 571 (MH+).

### Intermediate 7: 4-{4-[3-(6-Methylpyridin-2-yl)-1-trityl-1H-pyrazol-4-yl]pyridin-2-yl}-benzoic acid

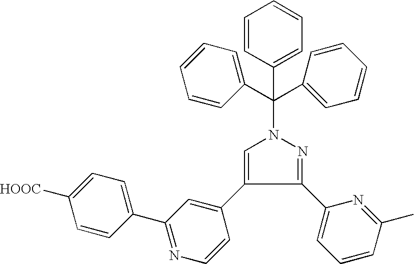

Intermediate 4 (1 g, 1.8 mmol) and 4-carboxybenzene boronic acid (0.36 g, 2.52 mmol) were coupled and treated as described for intermediate 6 to afford the title compound as a white solid (600 mg, 61%) containing the 2-trityl isomer as a minor component; [APCI MS] m/z 599 (MH+).

### Intermediate 8: 4-{4-[3-(6-methylpyridin-2-yl)-1-trityl-1H-pyrazol-4-yl]pyridin-2-yl}benzaldehyde

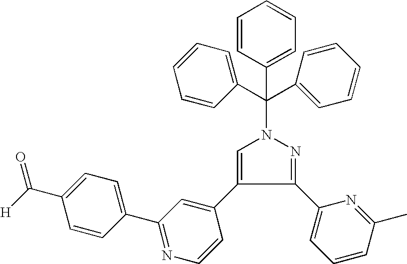

Intermediate 4 (1 g, 1.8 mmol) and 4-formylphenylboronic acid (0.35 g, 2.3 mmol) were coupled and treated as described for intermediate 6 to afford the title compound as a grey solid (1 g, 96%) containing the 2-trityl isomer as a minor component; [APCI MS] m/z 583 (MH+).

### Intermediate 9: 2-(4-bromophenyl)-4-[3-(6-methylpyridin-2-yl)-1-(triphenylmethyl)-1H-pyrazol-4-yl]pyridine

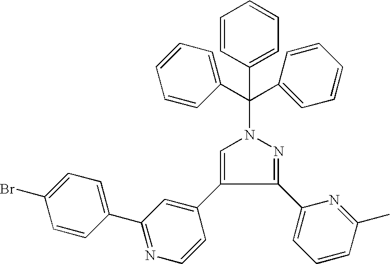

Intermediate 4 (2 g, 3.6 mmol) and 4-bromophenylboronic acid (0.755 g, 3.78 mmol) were coupled and treated as described for intermediate 6 to afford the title compound as a white solid (2.1 g, 92%) containing the 2-trityl isomer as a minor component; [APCI MS] m/z 633/635 (MH+).

### Intermediate 10: (4-{4-[3-(6-methylpyridin-2-yl)-1-(triphenylmethyl)-1H-pyrazol-4-yl]-pyridin-2-yl}phenyl)amine

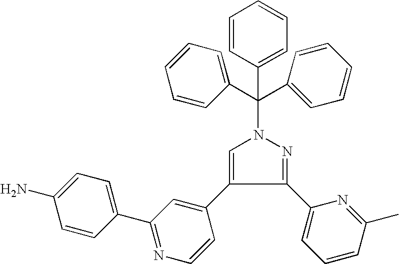

Intermediate 4 (1 g, 1.8 mmol) and 4-(4,4,5,5-tetramethyl-1,3,2-dioxaborolan-2-yl)aniline (0.512 g, 2.3 mmol) were coupled and treated as described for intermediate 6 to afford the title compound as a yellow solid (1 g, 98%) containing the 2-trityl isomer as a minor component; [APCI MS] m/z: 570 (MH+).

### Intermediate 11: 4-{4-[5-methyl-3-(6-methylpyridin-2-yl)-1-(triphenylmethyl)-1H-pyrazol-4-yl]-2-pyridinyl}benzaldehyde

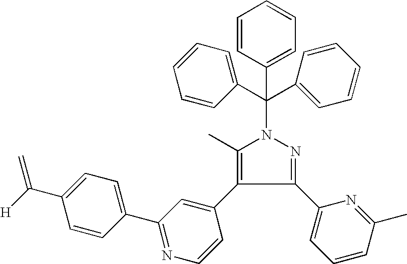

Intermediate 5 (1.5 g, 2.62 mmol) and 4-formylphenylboronic acid (0.48 g, 3.2 mmol) were coupled and treated as described for intermediate 6 to afford the title compound as a yellow oil (1.6 g, quantitative) containing the 2-trityl isomer as a minor component; [APCI MS] m/z 355 (MH+, loss of trityl).

### Intermediate 12: 4-bromophenyl-morpholine

To a solution of 4-phenyl-morpholine (18 g, 110.4 mmol) in ethanol (400 ml) cooled in an iced bath, was added dropwise bromine (5.95 ml, 116 mmol). The mixture was stirred at room temperature for 2 h and then poured into water. The solution was basified with NaOH 1N. The resulting precipitate was filtered and dried. After crystallisation from diisopropyl ether, the title compound was obtained as white crystals (15 g, 56.13%); m.p. 126-128° C.

### Intermediate 13: 4-(2-(pyrrolidin-1-yl)-ethoxy)-iodobenzene

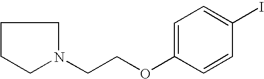

To a solution of 4-iodo-phenol (6 g, 27.3 mmol) in acetone (200 ml) were added caesium carbonate (22.2 g, 68.4 mmol) and N-(2-chloroethyl)-pyrrolidine.hydrochloride (7 g, 41 mmol) and the mixture was heated under reflux for 4 h and then poured into water. After extraction with CH₂Cl₂, the organic phase was dried over Na₂SO₄ and concentrated under reduced pressure. The title compound was obtained as a red oil (8 g, 92.53%); ¹H NMR (300 MHz, CDCl₃) 8 ppm: 7.5 (d, 2H), 6.65 (d, 2H), 4 (t, 2H), 2.8 (t, 2H), 2.55 (m, 4H), 1.75 (m, 4H).

### Intermediate 14: 4-(4,4,5,5-tetramethyl-[1,3,2]dioxaborolan-2-yl)-N-(tetrahydropyran-4-yl)-benzamide

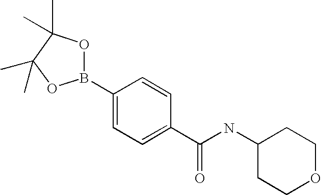

4-(4,4,5,5-tetramethyl-[1,3,2]dioxaborolan-2-yl)-benzoic acid (70.16 g, 0.28 mol) was treated with SOCl₂ (2 vol.) and the reaction mixture was stirred to reflux for 2 hours. After evaporation, the residue was diluted in toluene and poured into a solution at 10° C. of tetrahydro-pyran-4-ylamine (34.34 g, 0.339) and triethylamine (79 mL, 0.57 mol) in CH₂Cl₂. The reaction mixture was stirred at room temperature for 2 days and water (490 mL) was added to give a precipitate which was filtered and washed with EtOAc. After purification by flash chromatography using CH₂Cl₂/MeOH (95:5), the title compound was obtained as a solid (17.02 g, 18%); ¹H NMR (400 MHz, CDCl₃, ppm) δ ppm: 7.85 (d, 2H), 7.72 (d, 2H), 5.98 (m, 1H), 4.20 (s, 1H), 3.99 (m, 2H), 3.35 (t, 2H), 2.01 (d, 2H), 1.57 (m, 2H), 1.35 (s, 12H).

### Intermediate 15: 1-ethyl-4-[(4-(4,4,5,5-tetramethyl-[1,3,2]dioxaborolan-2-yl)-phenyl)carbonyl]-piperazine

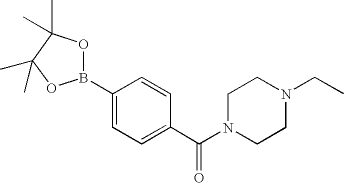

To a solution of 4-(4,4,5,5-tetramethyl-[1,3,2]dioxaborolan-2-yl)-benzoic acid (8.24 g, 33.22 mmol) in CH₂Cl₂ (50 ml) were added N-ethylpiperazine (5.1 ml, 39.87 mmol), HOBT (5.4 g, 39.87 mmol), EDCI (7.6 g, 39.87 mmol) and triethylamine (6.95 ml, 49.84 mmol) and the mixture was stirred at room temperature for 48 hours and then poured into water. After extraction with CH₂Cl₂, the organic phase was dried over Na₂SO₄ and concentrated under reduced pressure. The residue was purified by chromatography on silica gel (CH₂Cl₂/MeOH, 95:5) to afford the title compound as a pale yellow oil which crystallised (9.64 g, 84%); [APCI MS] m/z 345 (MH⁺).

### Intermediate 16: 4-[4-(4,4,5,5-tetramethyl-1,3,2-dioxaborolan-2-yl)phenyl]-morpholine

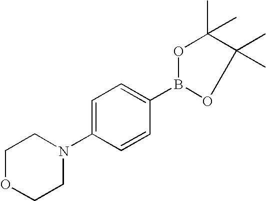

To a solution of intermediate 12 (15 g, 62 mmol) in dioxane (400 ml) were added 4,4,5,5-tetramethyl-1,3,2-dioxaborolane (9.9 ml, 68 mmol), dichlorobis(triphenylphosphine)palladium(II) (2.17 g, 3.1 mmol) and triethylamine (25.8 ml, 186 mmol) and the mixture was heated under reflux for 24 h and then poured into water. After extraction with CH₂Cl₂, the organic phase was dried over Na₂SO₄ and concentrated under reduced pressure. The residue was purified by chromatography on silica gel eluting with CH₂Cl₂, and after trituration with pentane, the title compound was obtained as a brown solid (15 g, 83.74%); m.p. 114-116° C.

### Intermediate 17: 1-[2-(pyrrolidin-1-yl)-ethoxy]-4-[4,4,5,5-tetramethyl-1,3,2-dioxaborolan-2-yl]-benzene

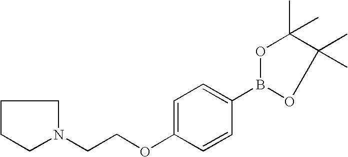

Intermediate 13 (8 g, 25.24 mmol) and 4,4,5,5-tetramethyl-1,3,2-dioxaborolane (4 ml, 27.6 mmol) were reacted as described for intermediate 16 to afford, after chromatography on silica gel (CH₂Cl₂/MeOH, 9:1), the title compound as a solid (8 g, 99.99%); m.p. 160-164° C.

### Intermediate 18: 1-[(morpholin-4-yl)carbonylmethyloxy]-4-[4,4,5,5-tetramethyl-1,3,2-dioxaborolan-2-yl]-benzene

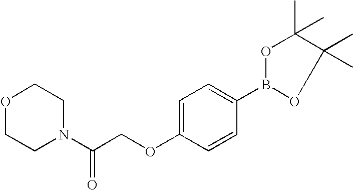

To a solution of 4-[4,4,5,5-tetramethyl-1,3,2-dioxaborolan-2-yl]-phenol (6.6 g, 30 mmol) in CH₃CN were added potassium carbonate (12.42 g, 90 mmol) and N-(chloroacethyl)-morpholine (4.89 g, 30 mmol) and the mixture was heated under reflux for 3 hours and then concentrated under reduced pressure. The residue was treated with water and extracted with EtOAc. The organic phase was dried over Na₂SO₄, and concentrated. After trituration with hexane, the title compound was obtained as a grey solid (9.5 g, 91%); m.p. 112° C.; [APCI MS] m/z 348 (MH+).

### Intermediate 19: 2-bromo-N-methoxy-N-methyl-4-pyridinecarboxamide

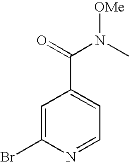

To a suspension of 2-bromo-4-pyridinecarboxylic acid (23.5 g, 116 mmol) in CH₂Cl₂ (600 mL) were added under nitrogen HOBT (17.3 g, 128 mmol), EDCI (24.5 g, 128 mmol), triethylamine (46.85 g, 464 mmol) and N,O-dimethylhydroxylamine hydrochloride (17.02 g, 175 mmol). The reaction mixture was stirred at room temperature for 3 h and then partitioned between water and CH₂Cl₂. The organic phase was dried over Na₂SO₄, filtered and evaporated under reduced pressure to afford the title compound as a white solid (17 g, 59%); [APCI MS] m/z 246 (MH+).

### Intermediate 20: 1-[2-bromo-pyridin-4-yl]-2-[6-methyl-pyridin-2-yl]-ethanone

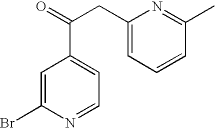

2,6-Lutidine (4.28 g; 40 mmol) was dissolved in dry THF (100 mL) under nitrogen and the solution was cooled to −30° C. 2.5M n-Butyllithium in hexanes (16 mL; 40 mmol) was added at −30° C., then the mixture was stirred 1.5 h at room temperature before being cooled to −30 to −40° C. A solution of intermediate 19 (4.9 g; 20 mmol) in dry THF (20 mL) was added at −40° C. and the reaction stirred for 2 h. Saturated aqueous ammonium chloride was added and the mixture was extracted with EtOAc. The organic phase was dried over Na₂SO₄, filtered and evaporated under reduced pressure. The residue was purified by chromatography on silica gel (CH₂Cl₂/MeOH, 99:1) to give the title compound (3.42 g; 58%) as a yellow solid; m.p. 126° C.; [APCI MS] m/z: 292 (MH⁺).

### Intermediate 21: 2-(6-methylpyridin-2-yl)-1-[2-(4-((tetrahydropyran-4-yl)-aminocarbonyl)phenyl)-pyridin-4-yl]-ethanone

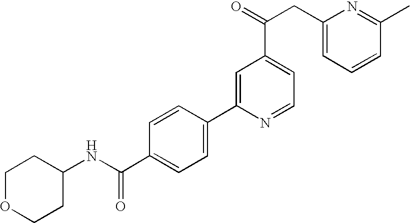

To a solution of intermediate 20 (1 g, 3.43 mmol) in DME (40 ml) were added intermediate 14 (1.25 g, 3.78 mmol), tetrakis(triphenylphosphine)palladium(0) (0.1 g) and Na₂CO₃ (solution 2M, 7.5 ml) and the mixture was heated under reflux for 1 h and then poured into water. After extraction with CH₂Cl₂, the organic phase was dried over Na₂SO₄, and concentrated under reduced pressure. The title compound was obtained as a yellow solid (1.4 g, 98.17%); m.p. 210° C.; [APCI MS] m/z 416 (MH⁺).

### Intermediate 22: 2-(6-methylpyridin-2-yl)-1-[2-(4-((1-ethyl-piperazin-4-yl)carbonyl)phenyl)-pyridin-4-yl]-ethanone

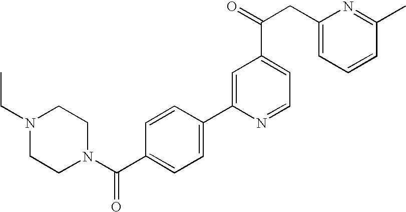

Intermediate 20 (1 g, 3.43 mmol) and intermediate 15 (1.28 g, 3.78 mmol) were reacted as was described for intermediate 21 to give the title compound as a yellow solid (0.95 g, 64.59%); m.p. 90° C.; [APCI MS] m/z 429 (MH⁺).

### Intermediate 23: 2-(6-methylpyridin-2-yl)-1-[2-(4-(morpholin-4-yl)phenyl)-pyridin-4-yl]-ethanone

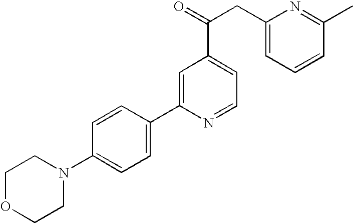

Intermediate 20 (1 g, 3.43 mmol) and intermediate 16 were reacted as was described for intermediate 21 to give the title compound as a yellow solid (1.2 g, 84%); m.p. 114° C.; [APCI MS] m/z 374 (MH⁺).

### Intermediate 24: 2-(6-methylpyridin-2-yl)-1-[2-(4-(2-(pyrolidin-1-yl)-ethoxy)-phenyl)-pyridin-4-yl]-ethanone

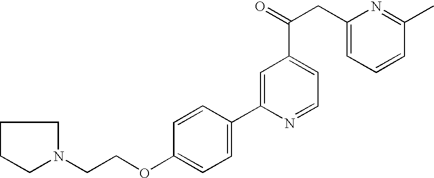

Intermediate 20 (1 g, 3.43 mmol) and intermediate 17 (1.2 g, 3.78 mmol) were reacted as was described for intermediate 21 to give, after chromatography on silica gel (CH₂Cl₂/MeOH, 9:1), the title compound as a yellow oil (0.8 g, 58%); [APCI MS] m/z 402 (MH⁺).

### Intermediate 25: 2-(6-methylpyridin-2-yl)-1-[2-(4-((morpholin-4-yl)-carbonylmethyloxy)-phenyl)-pyridin-4-yl]-ethanone

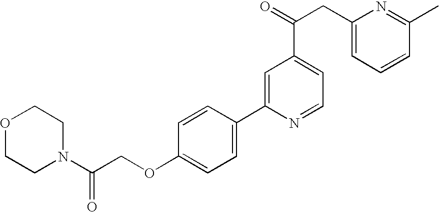

Intermediate 20 (1 g, 3.43 mmol) and intermediate 18 (1.45 g, 4.12 mmol) were reacted as was described for intermediate 21 to give, after chromatography on silica gel (CH₂Cl₂/MeOH, 98/2), the title compound as a yellow gum (1 g, 67.52%); [APCI MS] m/z 432 (MH⁺).

### Intermediate 26: 2-(6-methylpyridin-2-yl)-1-[2-(4-formylphenyl)-pyridin-4-yl]-ethanone

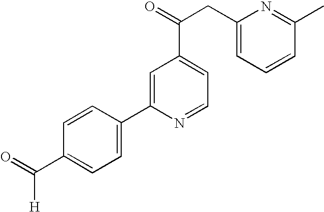

Intermediate 20 (2 g, 7 mmol) and 4-formylphenylboronic acid (1.34 g, 9 mmol) were reacted as was described for intermediate 21 to give the title compound as a yellow solid (2.1 g, 96.69%); m.p. 118° C.; [APCI MS] m/z 317 (MH⁺).

### Intermediate 27: 2-(6-methylpyridin-2-yl)-1-[2-(4-((morpholin-4-yl)methyl)-phenyl)-pyridin-4-yl]-ethanone

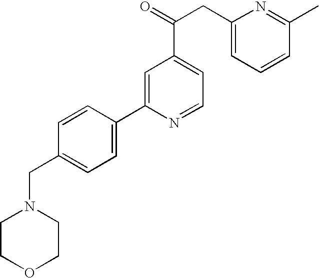

To a solution of intermediate 26 (0.984 g, 3 mmol) in 1,2-dichloroethane (40 ml) were added morpholine (0.34 g, 3.9 mmol), sodium triacetoxyborohydride (0.826 g, 3.9 mmol) and acetic acid (0.216 g, 3.6 mmol) and the mixture was stirred at room temperature for 3 h and then poured into water. After extraction with CH₂Cl₂, the organic phase was dried over Na₂SO₄ and concentrated under reduced pressure. The title compound was obtained as an oil (1.1 g, 91%); [APCI MS] m/z 388 (MH⁺).

### Intermediate 28:1-methyl-4-hydroxymethyl-imidazole

To a suspension of 1-methyl-imidazole-4-carboxylic acid (11.4 g, 90 mmol) in THF (500 ml) at 0° C., was added dropwise LiAlH₄ (solution 1M in THF, 117 ml, 117 mmol) and the mixture was stirred at room temperature overnight and then at 50° C. for 1 hour. Water (3 ml) was added followed by Na₂SO₄, and the resulting precipitate was filtered though a celite pad. The filtrate was concentrated under reduced pressure to afford the title compound as a solid (8 g, 78.95%); ¹H NMR (300 MHz, CDCl₃) δ ppm: 7.25 (s, 1H), 6.70 (s, 1H), 5.25 (m,1H), 4.40 (s, 2H), 3.45 (s, 3H).

### Intermediate 29: 1-methyl-4-chloromethyl-imidazole, hydrochloride

To a solution of intermediate 28 (5 g, 44.64 mmol) in CH₂Cl₂ (10 ml) at 0° C. was added dropwise thionyl chloride (50 ml) and then the mixture was stirred at room temperature overnight and then under reflux for 3 hours. On cooling the mixture was concentrated under reduced pressure. The residue was treated with diethyl ether and the resulting precipitate was filtered and dried. The title compound was obtained as a brown solid (4 g, 53.81%); ¹H NMR (300 MHz, d⁶-DMSO) δ ppm: 9.25 (s, 1H), 7.8 (s, 1H), 4.95 (s, 2H), 3.9 (s, 3H).

### EXAMPLES

### Example 1

### 2-{4-[(1-methyl-1H-imidazol-4-yl)methoxy]phenyl}-4-[3-(6-methylpyridin-2-yl)-1H-pyrazol-4-yl]pyridine

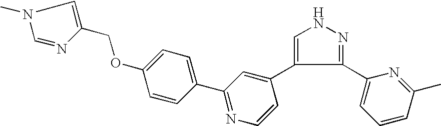

To a solution of intermediate 6 (4 g, 7 mmol) in DMF (80 ml) cooled in an ice bath, was added portion-wise sodium hydride (0.6 g, 3 eq, 21 mmol) and the mixture then stirred at room temperature for 30 mins. Intermediate 29 (1.6 g, 10 mmol) was added and the mixture stirred at room temperature overnight and then poured into water and extracted with CH₂Cl₂. The organic layer was dried over Na₂SO₄ and filtered. Evaporation of the solvent in vacuo gave a crude oil which was purified by chromatography on silica gel (CH₂Cl₂/MeOH 97:3) to give the trityl compound as an oil (3 g). This compound was dissolved in methanol (60 ml) and HCl (1N, 40 ml) and the solution was heated under reflux for 2 hours and then concentrated in vacuo. The residue was dissolved in water and washed with CH₂Cl₂. The aqueous layer was basified with NaOH (1N) and extracted with CH₂Cl₂. The organic extract was washed with water, dried over Na₂SO₄, filtered and evaporated to give a solid which was crystallised from EtOH to give the title compound as white crystals (1.1 g, 37%); m.p. 191° C.; TOF MS ES⁺ exact mass calculated for C₂₅H₂₂N₆O₁: 423.1933 (MH+). Found: 423.1928 (MH+).

### Example 2

### 2-[4-(Ethylsulfonyl)phenyl]-4-[3-(6-methylpyridin-2-yl)-1H-pyrazol-4-yl]pyridine

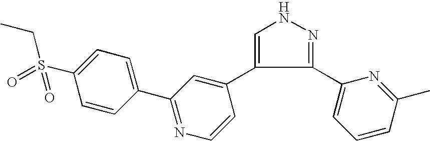

To a solution of intermediate 4 (0.5 g, 0.9 mmol) in a mixture of DME (18 ml) and water (9 ml) was added 4-(ethylsulfonyl)phenyl boronic acid (1.3 eq, 0.25 g, 1.17 mmol), tetrakis triphenylphosphine palladium (0.05 g) and Na₂CO₃ (3 eq, 0.28 g, 2.69 mmol) and the reaction mixture was heated under reflux overnight. The cooled mixture was poured into ice and extracted with CH₂Cl₂. The organic layer was washed with water, dried over Na₂SO₄ and filtered. Evaporation of the solvent in vacuo gave an oil which was dissolved in MeOH (30 ml) and HCl (1N, 20 ml). The solution was heated under reflux for 3 hours and then concentrated under reduced pressure. The residue was dissolved in water and washed with CH₂Cl₂. The aqueous layer was basified with NaOH (1N) and extracted with CH₂Cl₂. The organic extract was washed with water, dried over Na₂SO₄, filtered and evaporated under reduced pressure. After chromatography on silica gel (CH₂Cl₂/MeOH, 95/5) and crystallisation from DMF, the title compound was obtained as white crystals (166 mg, 45.7%); m.p. 244° C.; [APCI MS] m/z 405 (MH+).

### Example 3

### 4-[3-(6-methylpyridin-2-yl)-1H-pyrazol-4-yl]-2-[4-(pyrrolidin-1-ylmethyl)phenyl]pyridine

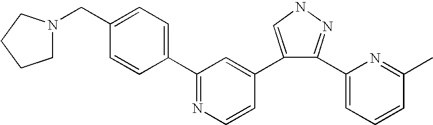

To a solution of intermediate 8 (0.29 g, 0.5 mmol) and pyrrolidine (4 eq, 0.142 g) in dry dichloroethane (20 mL) was added acetic acid (1.5eq, 0.05 g) followed by sodium triacetoxyborohydride (2eq, 0.224 g). The mixture was stirred at room temperature overnight, diluted with water, extracted with CH₂Cl₂ and dried over Na₂SO₄. The solvent was removed under reduced pressure and the resulting product was treated with a mixture of MeOH/HCl 1N (3:2, 50 ml) at reflux for 3 h. The reaction mixture was concentrated to dryness to give a residue which was dissolved in water and washed with CH₂Cl₂. The aqueous phase was basified with 1N NaOH, extracted with CH₂Cl₂ and dried over Na₂SO₄. Concentration to dryness gave a solid which was precipitated from a mixture CH₂Cl₂/hexane to give the title compound as a solid (0.095 g, 48%); ¹H NMR (300 MHz, CDCl₃ ppm δ 8.62 (d, 1H); 7.88 (d, 2H); 7.71 (d, 2H); 7.50-7.39 (m, 3H); 7.26-7.20 (m,2H); 7.05 (d, 1H); 3.76 (brs, 2H); 2.79-2.56 (m, 4H); 2.53 (s, 3H); 1.91-1.75 (m, 4H); TOF MS ES⁺ exact mass calculated for C₂₅H₂₅N₆ (MH+): 396.2188. Found: 396.2174 (MH+).

### Example 4

### 4-(4-{4-[3-(6-methylpyridin-2-yl)-1H-pyrazol-4-yl]pyridin-2-yl}benzyl)morpholine

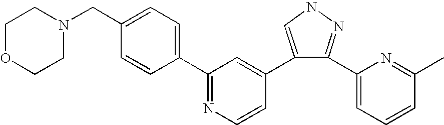

Intermediate 8 (0.582 g, 1.5 mmol) and morpholine (0.6 g, 6 mmol) were coupled and treated as was described for example 3 to give the title compound (0.4 g, 65%); ¹H NMR (300 MHz, CDCl₃) δ 8.70 (d,1H); 7.98 (d, 2H); 7.81 (d, 2H); 7.58-7.41 (m, 3H); 7.37-7.30 (m, 2H); 7.14 (1H, d); 3.89-3.70 (m, 4H); 3.62 (brs, 2H); 2.62 (s, 3H); 2.60-2.47 (m, 4H); TOF MS ES⁺ exact mass calculated for C₂₅H₂₅N₅O: 412.2137 (MH+). Found: 412.2150 (MH+).

### Example 5

### 2-methoxy-N-methyl-N-(4-{4-[3-(6-methylpyridin-2-yl)-1H-pyrazol-4-yl]pyridin-2-yl}benzyl)ethanamine

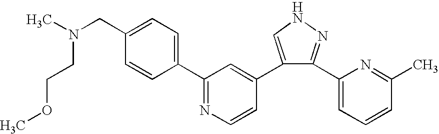

Intermediate 8 (g, mmol) and N-methyl-2-(methyloxy)ethanamine (0.107 g, 1.2 mmol) were coupled and treated as was described for example 3 to give the title compound as colourless oil (0.3 g, 72.3%); ¹H NMR (300 MHz, CDCl₃) δ 8.59 (d, 1H); 7.83 (d, 2H); 7.71 (s, 1H); 7.66 (s, 1H); 7.47-7.29 (m, 3H); 7.24-7.18 (m, 2H); 7.03 (d, 1H); 3.59-3.50 (m, 2H); 3.45 (brt, 2H); 3.25 (s, 3H); 2.56 (brt, 3H); 2.60-2.47 (m, 4H); 2.50 (s, 3H); [APCI MS] m/z 414 (MH+).

### Example 6

### 2-{4-[(4-methoxypiperidin-1-yl)methyl]phenyl}-4-[3-(6-methylpyridin-2-yl)-1H-pyrazol-4-yl]pyridine

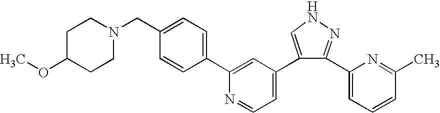

Intermediate 8 (0.67 g, 1.15 mmol) and 4-(methyloxy)piperidine hydrochloride (0.174 g, 1.15 mmol) were coupled and treated as was described for example 3 to give the title compound as a white powder (0.195 g, 38.6%); ¹H NMR (300 MHz, CDCl₃) δ 8.64 (d, 1H); 7.88 (d, 2H); 7.76 (s, 1H); 7.72(s, 1H); 7.47 (t, 1H); 7.44-7.35 (m, 2H); 7.29-7.23 (m, 2H); 7.09 (d, 1H); 3.61-3.50 (m, 2H); 3.29 (s, 3H); 3.26-3.14 (m, 1H); 2.81-2.66 (m, 2H); 2.56 (s, 3H); 2.30-2.08 (m, 2H); 2.01-1.82 (m, 2H); 1.69-1.52 (m, 2H); TOF MS ES⁺ exact mass calculated for C₂₇H₂₉N₅O: 440.2450 (MH+). Found: 440.2438 (MH+).

### Example 7

### 4-(4-{4-[5-methyl-3-(6-methylpyridin-2-yl)-1H-pyrazol-4-yl]pyridin-2-yl}benzyl)morpholine

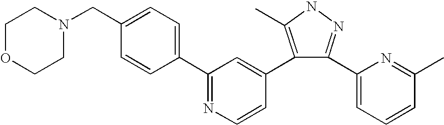

Intermediate 11 (1.6 g, 2.7 mmol) and morpholine (0.35 g, 4 mmol) were coupled and treated as was described for example 3 to give the title compound as a white powder (0.3 g, 26.14%); ¹H NMR (300 MHz, CDCl₃) δ 8.66 (d, 1H); 7.86 (d, 2H); 7.64 (s, 1H); 7.44-7.30 (m, 3H); 7.15-7.11 (m, 1H); 7.03-6.89 (m, 2H); 3.74-3.60 (m, 4H); 3.51 (brs, 2H); 2.50 (s, 3H); 2.40-2.34 (m, 4H); 2.25 (s, 3H); [APCI MS] m/z 426 (MH+).

### Example 8

### 4-(4-{4-[3-(6-methylpyridin-2-yl)-1H-pyrazol-4-yl]pyridin-2-yl}benzoyl)morpholine

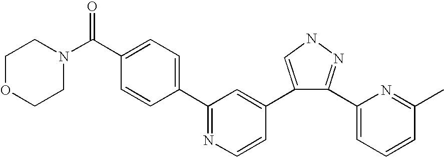

To a solution of intermediate 7 (0.2 g, 0.34 mmol, 1 eq.) in CH₂Cl₂ (50 mL) were added morpholine (0.035 g, 0.4 mmol), HOBT (0.59 g, 1.3eq.), EDCI (0.83 g, 1.3eq.) and Et₃N (0.04 g, 2.3eq.) and the reaction mixture was stirred at room temperature overnight. The reaction was hydrolysed and extracted with CH₂Cl₂. The solvent was removed under reduced pressure. The residue was treated with MeOH/HCl 1N (3/2, 30 ml) at reflux for 1 h. After removal of the solvent under reduced pressure, the residue was dissolved into water and washed with CH₂Cl₂. The aqueous phase was basified with NaOH 1N and extracted with CH₂Cl₂. The organic phase was dried, filtered and evaporated to dryness to give a crude solid which was precipitated with a mixture of CH₂Cl₂/Hexane to give the title compound as a (0.075 g; 52%); ¹H NMR (300 MHz, CDCl₃) δ 8.63 (d, 1H); 7.95 (d, 2H); 7.76 (s, 2H); 7.52-7.42 (m, 3H); 7.27 (d, 2H); 7.12-7.01 (m, 1H); 3.50-3.32 (m, 8H); 2.51 (brs, 3H); TOF MS ES⁺ exact mass calculated for C₂₅H₂₃N₅O₂: 426.1930 (MH+). Found: 426.1931 (MH+).

### Example 9

### 4-{4-[3-(6-methylpyridin-2-yl)-1H-pyrazol-4-yl]pyridin-2-yl}-N-(tetrahydro-2H-pyran-4-yl)benzamide

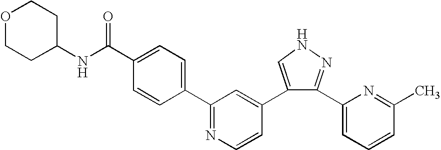

Intermediate 7 (0.4 g, 0.67 mmol) and 4-aminotetrahydropyran (0.081 g, 0.8 mmol) were reacted as was described for example 8 to give the title compound as a (0.2 g, 68%); m.p. 148° C.; TOF MS ES⁺ exact mass calculated for C₂₆H₂₅NSO₂: 440.2086 (MH+). Found: 440.2060 (MH+).

### Example 10

### N-(4-{4-[3-(6-methylpyridin-2-yl)-1H-pyrazol-4-yl]pyridin-2-yl}phenyl)-2-morpholin-4-ylacetamide

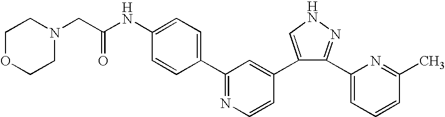

Intermediate 10 (0.38 g, 0.67 mmol) and 4-morpholinylacetic acid hydrochloride (0.156 g, 0.86 mmol) were reacted as was described for example 8 to give the title compound as an off-white solid (0.115 g, 38%); ¹H NMR (300 MHz, CDCl₃) δ 9.10 (s, 1H); 8.58(d, 1H); 7.87 (d, 2H); 7.70 (s, 2H); 7.59 (d, 2H); 7.28-7.18 (m, 2H); 7.10-6.97 (m, 1H); 3.71 (t, 4H); 3.08 (s, 3H); 2.56 (t, 4H); 2.50 (s, 3H); TOF MS ES⁺ exact mass calculated for C₂₆H₂₆N₆O₂: 455.2195 (MH+). Found: 455.2195 (MH+).

### Example 11

### 4-{4-[3-(6-methylpyridin-2-yl)-1H-pyrazol-4-yl]pyridin-2-yl}benzonitrile

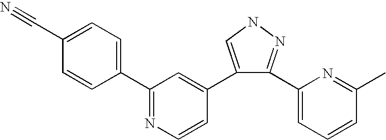

Intermediate 4 (0.37 g, 0.67 mmol) and 4-cyanophenyl boronic acid (0.128 g, 0.87 mmol) were coupled and treated as described for example 2 to afford the title compound as an off-white solid (0.118 g, 64%); m.p. 198° C.; TOF MS ES⁺ exact mass calculated for C₂₁H₁₅N₅: 338.1406 (MH+). Found: 338.1408 (MH+).

### Example 12

### 4-[3-(6-methylpyridin-2-yl)-1H-pyrazol-4-yl]-2-[4-trifluoromethoxy)phenyl]pyridine

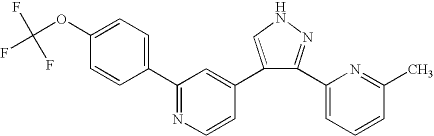

Intermediate 4 (0.37 g, 0.67 mmol) and 4-trifluoromethoxyphenyl boronic acid (0.179 g, 0.87 mmol) were coupled and treated as described for example 2 to afford the title compound as an white solid (0.185 g, 69.7%); m.p. 131° C.; TOF MS ES⁺ exact mass calculated for C₂₁H₁₅F₃N₄O: 397.1276 (MH+). Found: 397.1269 (MH+).

### Example 13

### 2-(4-chlorophenyl)-4-[3-(6-methylpyridin-2-yl)-1H-pyrazol-4-yl]pyridine

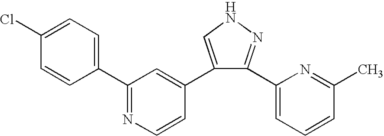

Intermediate 4 (0.556 g, 1 mmol) and 4-chlorophenyl boronic acid (0.184 g, 1.2 mmol) were coupled and treated as described for example 2 to afford the title compound as an white solid (0.037 g, 10.7%); m.p. 190° C.; TOF MS ES⁺ exact mass calculated for C₂₀H₁₅ClN₄: 347.1063 (MH+). Found: 347.1057 (MH+).

### Example 14

### 2-(4-methoxyphenyl)-4-[3-(6-methylpyridin-2-yl)-1H-pyrazol-4-yl]pyridine

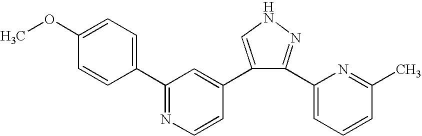

Intermediate 4 (0.278 g, 0.5 mmol) and 4-methoxyphenyl boronic acid (0.051 g, 0.6 mmol) were coupled and treated as described for example 2 to afford the title compound as an white solid (0.043 g, 25%); ¹H NMR (300 MHz, CDCl₃) δ 8.84(d, 1H); 8.10 (d, 2H); 7.96-7.91 (m, 2H); 7.80 (t, 1H); 7.66 (d, 1H); 7.49-7.36 (m, 2H); 7.17 (d, 2H); 4.04 (s, 3H); 2.65 (s, 3H); TOF MS ES⁺ exact mass calculated for C₂₁H₁₈N₄O: 343.16 (MH+). Found: 343.16 (MH+).

### Example 15

### 2-[4-(Methylsulfonyl)phenyl]-4-[3-(6-methylpyridin-2-yl)-1H-pyrazol-4-yl]pyridine

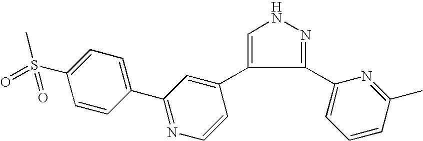

Intermediate 4 (0.8 g, 1.44 mmol) and 4-methylsulfonylphenyl boronic acid (0.316 g, 1.58 mmol) were coupled and treated as described for example 2 to afford the title compound as a white powder (0.14 g, 25%); m.p. 232-234° C.; ¹H NMR (300 MHz, CDCl₃) δ 8.84(d, 1H); 8.52 (d, 2H); 8.44 (s, 1H); 8.26 (d, 2H); 8.01 (t, 1H); 7.89-7.68 (m, 2H); 7.50 (d, 1H); 3.51 (s, 3H); 2.64 (s, 3H); [APCI MS] m/z 391 (MH+).

### Example 16

### 4-(4-{4-[3-(6-methylpyridin-2-yl)-1H-pyrazol-4-yl]pyridin-2-yl}phenyl)morpholine

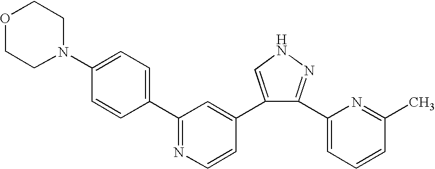

Step 1: To a solution of intermediate 9 (0.6339, 1 mmol) in toluene (10 ml) were added morpholine (0.3489, 4 mmol, 4eq), Pd₂(dba)₃ (0.045 g, 0.049 mmol, 0.05eq), binap (0.062 g, 0.1 mmol, 0.1 eq) and t-BuOK (0.134 g, 1.4 mmol, 1.4eq) and the reaction mixture was refluxed for 5 hours. The mixture was then poured into ice and extracted with EtOAc. The organic phase was washed with water and dried over Na₂SO₄. Concentration to dryness gave a crude product that was purified by chromatography on silica gel (CH₂Cl₂/CH₃OH 98:2) to afford 4-(4-{4-[3-(6-methyl-2-pyridinyl)-1-(triphenylmethyl)-1H-pyrazol-4-yl]-2-pyridinyl}phenyl)morpholine.

Step 2: The product from step 1 was treated with a mixture of MeOH/HCl 1N (3:2, 50 ml) under reflux for 2 hours. The reaction mixture was poured into water and extracted with CH₂Cl₂. The aqueous phase was basified with NaOH 1N and extracted with CH₂Cl₂. The organic phase was washed with water, dried and evaporated to dryness to give a crude product which was precipitated from a mixture of CH₂Cl₂/hexane to afford the title compound as a yellow solid (0.31 g, 78%); TOF MS ES⁺ exact mass calculated for C₂₄H₂₃N₅O: 398.1981 (MH+). Found 398.1961 (MH+).

### Example 17

### 2-[4-(2-methyl-1H-imidazol-1-yl)phenyl]-4-[3-(6-methylpyridin-2-yl)-1H-pyrazol-4-yl]pyridine

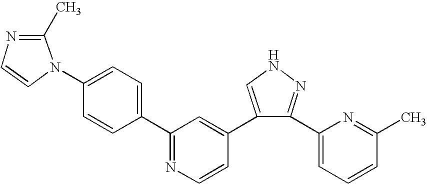

Intermediate 9 (0.308 g, 0.5 mmol) and 2-methyl-1H-imidazole (0.82 g, 1 mmol) were treated as described for example 16 to give the title compound as a white solid (0.013 g, 6%); m.p. 128° C.; ¹H NMR (300 MHz; CDCl₃) δ ppm: 8.64 (d, 1H), 8.02 (d, 2H), 7.75 (d, 2H), 7.48 (t, 1H), 7.37-7.20 (m, 4H), 7.19 (s, 1H), 7.08 (d, 1H), 6.99 (d, 2H), 2.52 (s, 3H), 2.35 (s, 3H).

### Example 18

### 3-[2-(4-((tetrahydropyran-4-yl)aminocarbonyl)phenyl)pyridin-4-yl]-4-[6-methylpyridin-2-yl]-1H-pyrazole

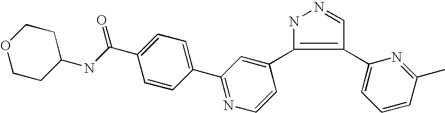

To a solution of intermediate 21 (0.6 g, 1.44 mmol) in DMF (10 ml) and acetic acid (0.2 ml, 3.47 mmol) was added DMF.DMA (0.258 g, 2.16 mmol) and the mixture was stirred at room temperature for 2 h. Hydrazine hydrate (3 ml) was added and the mixture was stirred at room temperature overnight, then was heated at 40° C. for 2 h and then poured into water. The aqueous phase was extracted with CH₂Cl₂, the organic phase dried over Na₂SO₄ and concentrated under reduced pressure. The residue was purified by chromatography on silica gel, eluting with CH₂Cl₂/MeOH (95/5). After crystallisation from EtOAc, the title compound was obtained as white crystals (0.2 g, 31.51%); mp: 170° C.; TOF MS ES⁺ exact mass calculated for C₂₆H₂₅N₅O₂: 440.2086 (MH+). Found 440.2065 (MH+).

### Example 19

### 3-[2-(4-((1-ethyl-piperazin-4-yl)carbonyl)phenyl)pyridin-4-yl]-4-[6-methylpyridin-2-yl]-1H-pyrazole

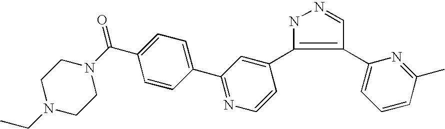

Intermediate 22 (0.428 g, 1 mmol) was reacted as described for example 18 to afford, after precipitation from CH₂Cl₂/hexane, the title compound as a yellow solid (0.045 g, 9.95%); m.p. 126° C.; TOF MS ES⁺ exact mass calculated for C₂₇H₂₈N₆O: 453.2403 (MH+) found 453.2394 (MH+).

### Example 20

### 3-[2-(4-(morpholin-4-yl)phenyl)pyridin-4-yl]-4-[6-methylpyridin-2-yl-1H-pyrazole

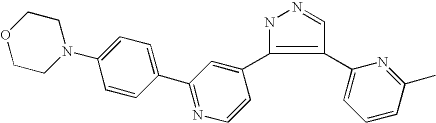

Intermediate 23 (0.373 g, 1 mmol) was reacted as described for example 18, to afford after crystallisation from EtOAc, the title compound as white crystals (0.275 g, 69%); m.p. 210° C.; TOF MS ES⁺ exact mass calculated for C₂₄H₂₃N₅O: 398.1981 (MH+). Found 398.1955 (MH+).

### Example 21

### 3-[2-(4-((morpholin-4-yl)methyl)phenyl)pyridin-4-yl-4-[6-methylpyridin-2-yl]-1H-pyrazole

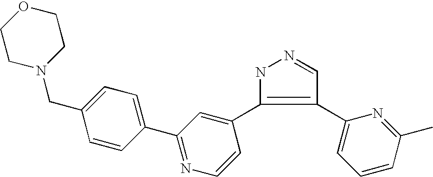

Intermediate 27 (0.387 g, 1 mmol) was reacted as described for example 18 to afford, after precipitation from CH₂Cl₂/hexane, the title compound as a white solid (0.296 g, 72%); m.p. 128° C.; TOF MS ES⁺ exact mass calculated for C₂₅H₂₅N₅O: 412.2137 (MH+). Found 412.2120 (MH+).

### Example 22

### 3-[2-(4-(2-(pyrolidin-1-yl)ethoxy)phenyl)pyridin-4-yl]-4-[6-methylpyridin-2-yl]-1H-pyrazole

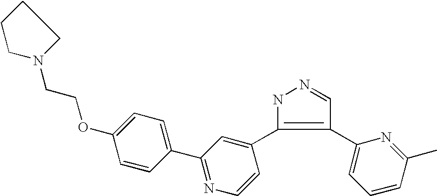

Intermediate 24 (0.4 g, 1 mmol) was reacted as described for example 18 to afford, after precipitation from CH₂Cl₂/hexane, the title compound as a white solid (0.142 g, 33.4%); m.p. 96° C.; TOF MS ES⁺ exact mass calculated for C₂₆H₂₇N₅O: 426.2294 (MH+). Found 426.2254 (MH+).

### Example 23

### 3-[2-(4-((morpholin-4-yl)carbonylmethyloxy)phenyl)pyridin-4-yl]-4-[6-methylpyridin-2-yl]-1H-pyrazole

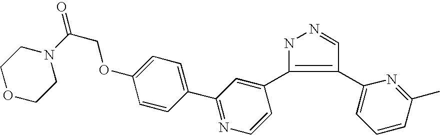

Intermediate 25 (0.431 g, 1 mmol) was reacted as described for example 18 to afford, after crystallisation from toluene, the title compound as white crystals (0.074 g, 16.3%); m.p. 170° C.; TOF MS ES⁺ exact mass calculated for C₂₆H₂₅N₅O₃: 456.2036 (MH+). Found 456.2012 (MH+).

Biology

The biological activity of the compounds of the invention may be assessed using the following assays:

Assay 1 (Cellular Transcriptional Assay)

The potential for compounds of the invention to inhibit TGF-β signalling may be demonstrated, for example, using the following in vitro assay.

The assay was performed in HepG2 cells stably transfected with the PAI-1 promoter (known to be a strong TGF-β responsive promoter) linked to a luciferase (firefly) reporter gene. The compounds were selected on their ability to inhibit luciferase activity in cells exposed to TGF-β. In addition, cells were transfected with a second luciferase (Renilla) gene which was not driven by a TGF-β responsive promoter and was used as a toxicity control.

96 well microplates were seeded, using a multidrop apparatus, with the stably transfected cell line at a concentration of 35000 cells per well in 200 μl of serum-containing medium. These plates were placed in a cell incubator.

18 to 24 hours later (Day 2), cell-incubation procedure was launched. Cells were incubated with TGF-β and a candidate compound at concentrations in the range 50 nM to 10 μM (final concentration of DMSO 1%). The final concentration of TGF-β (rhTGFβ-1) used in the test was 1 ng/mL. Cells were incubated with a candidate compound 15-30 mins prior to the addition of TGF-β. The final volume of the test reaction was 150 μl. Each well contained only one candidate compound and its effect on the PAI-1 promoter was monitored.

Columns 11 and 12 were employed as controls. Column 11 contained 8 wells in which the cells were incubated in the presence of TGF-β, without a candidate compound. Column 11 was used to determine the ‘reference TGF-β induced firefly luciferase value’ against which values measured in the test wells (to quantify inhibitory activity) were compared. In wells A12 to D12, cells were grown in medium without TGF-β. The firefly luciferase values obtained from these positions are representative of the ‘basal firefly luciferase activity’. In wells E12 to H12, cells were incubated in the presence of TGF-β and 500 μM CPO (Cyclopentenone, Sigma), a cell toxic compound. The toxicity was revealed by decreased firefly and renilla luciferase activities (around 50% of those obtained in column 11).

12 to 18 hours later (day 3), the luciferase quantification procedure was launched. The following reactions were performed using reagents obtained from a Dual Luciferase Assay Kit (Promega). Cells were washed and lysed with the addition of 10 μl of passive lysis buffer (Promega). Following agitation (15 to 30 mins), luciferase activities of the plates were read in a dual-injector luminometer (BMG lumistar). For this purpose, 50 μl of luciferase assay reagent and 50 μL of ‘Stop & Glo’ buffer were injected sequentially to quantify the activities of both luciferases. Data obtained from the measurements were processed and analysed using suitable software. The mean Luciferase activity value obtained in wells A11 to H11 (Column 11, TGF-βonly) was considered to represent 100% and values obtained in wells A12 to D12 (cells in medium alone) gave a basal level (0%). For each of the compounds tested, a concentration response curve was constructed from which an IC₅₀ value was determined graphically.

Assay 2 (Alk5 Fluorescence Polarization Assay)

Kinase inhibitor compounds conjugated to fluorophores, can be used as fluorescent ligands to monitor ATP competitive binding of other compounds to a given kinase. The increase in depolarization of plane polarized light, caused by release of the bound ligand into solution, is measured as a polarization/anisotropy value. This protocol details the use of a rhodamine green-labelled ligand for assays using recombinant GST-ALK5 (residues 198-503).

Assay buffer components: 62.5 mM Hepes pH 7.5 (Sigma H-4034), 1 mM DTT (Sigma D-0632), 12.5 mM MgCl₂ (Sigma M-9272), 1.25 mM CHAPS (Sigma C-3023).

Protocol: Solid compound stocks were dissolved in 100% DMSO to a concentration of 1 mM and transferred into column 1, rows A-H of a 96-well, U bottom, polypropylene plate (Costar #3365) to make a compound plate. The compounds were serially diluted (3-fold in 100% DMSO) across the plate to column 11 to yield 11 concentrations for each test compound. Column 12 contained only DMSO. A Rapidplate™-96 was used to transfer 1 μl of sample from each well into a 96-well, black, U-bottom, non-treated plate (Costar #3792) to create an assay plate.

ALK5 was added to assay buffer containing the above components and 1 nM of the rhodamine green-labelled ligand so that the final ALK5 concentration was 10 nM based on active site titration of the enzyme. The enzyme/ligand reagent (39 μl) was added to each well of the previously prepared assay plates. A control compound (1 μl) was added to column 12, rows E-H for the low control values. The plates were read immediately on a LJL Acquest fluorescence reader (Molecular Devices, serial number AQ1048) with excitation, emission, and dichroic filters of 485 nm, 530 nm, and 505 nm, respectively. The fluorescence polarization for each well was calculated by the Acquest reader and then imported into curve fitting software for construction of concentration response curves. The normalized response was determined relative to the high controls (1 μl DMSO in column 12, rows A-D) and the low controls (1 μl of control compound in column 12, rows E-H). An IC₅₀ value was then calculated for each compound

Using the above assays all Examples of the invention show ALK5 receptor modulator activity (having IC₅₀ values in the range of 1 to 100 nM) and TGF-β cellular activity (having IC₅₀ values in the range of 0.001 to 10 μM).

2-{4-(1-Methyl-imidazol-4-yl)methyloxy]-phenyl}4-(3-(6-methyl-pyridin-2-yl)-1H-pyrazol-4-yl)pyridine (Example 1) showed an ALK5 receptor modulator activity of 5 nM and TGF-β cellular activity of 34 nM.

3-[2-(4-(2-(Pyrolidin-1-yl)ethoxy)phenyl)pyridin-4-yl]-4-[6-methylpyridin-2-yl]-1H-pyrazole (Example 22) showed an ALK5 receptor modulator activity of 17 nM and TGF-β cellular activity of 38 nM.

1. A compound of formula (I), a pharmaceutically acceptable salt, solvate or derivative thereof; 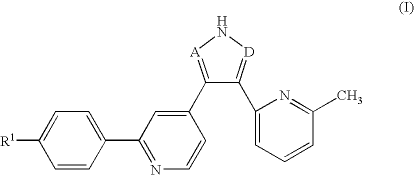
wherein
either a) A is C(R²) and D is N; or b) A is N and D is C(R²);
R¹ is selected from the list: hydrogen, C₁₋₆alkyl, C₁₋₆alkenyl, C₁₋₆alkoxy, halo, cyano, perfluoro C₁₋₆alkyl, perfluoroC₁₋₆alkoxy, —NR³R⁴, —(CH₂)ₙNR³R⁴, —O(CH₂)ₙOR⁵, —O(CH₂)ₙNR³R⁴, —O(CH₂)ₙHet, —CONR³R⁴, —CO(CH₂)ₙNR³R⁴, —SO₂R⁵, —SO₂NR³R⁴, —NR³SO₂R⁵, —NR³COR⁵, —NR³CO(CH₂)ₙNR³R⁴, Het and —O(CH₂)ₙCONR³R⁴;
R² is hydrogen or C₁₋₄alkyl;
R³ and R⁴ are independently hydrogen, C₁₋₆alkyl, Het or C₁₋₄alkoxyC₁₋₄alkyl; or R³ and R⁴ together with the nitrogen atom to which they are attached form a 3, 4, 5, 6 or 7-membered saturated or unsaturated ring which may contain one or more heteroatoms selected from N, S or O, and wherein the ring may be further substituted by one or more substituents selected from halo (such as fluoro, chloro, bromo), —CN, —CF₃, —OH, —OCF₃, C₁₋₆alkyl and C]-₆alkoxy;
R⁵ is hydrogen or C₁₋₆alkyl;
Het is a 5 or 6-membered C-linked heterocyclyl group which may be saturated, unsaturated or aromatic, which may contain one or more heteroatoms selected from N, S or O and which may be substituted by C₁₋₆alkyl; and
n is 1-4.

2. A compound according to claim 1 wherein R¹ is C₁₋₆alkoxy, halo, cyano, perfluoroC₁₋₆alkoxy, —NR³R⁴, —(CH₂)ₙNR³R⁴, —O(CH₂)ₙNR³R⁴, —O(CH₂)ₙHet, —CONR³R⁴, —SO₂R⁵, —NR³CO(CH₂)ₙNR³R⁴ Het or —O(CH₂)ₙCONR³R⁴.

3. A compound according to claim 1 wherein R³ and R⁴ are independently hydrogen, methyl, Het or C₁₋₄alkoxyC₁₋₄alkyl; or R and R⁴ together with the atom to which they are attached form a morpholine, piperidine, pyrrolidine or piperazine ring.

4. A compound according to claim 1 wherein either a) A is C(R²) and D is N; or b) A is N and D is C(R²);
R¹ is C₁₋₆alkoxy, halo, cyano, perfluoroC₁₋₆alkoxy, —NR³R⁴, —(CH₂)ₙNR³R⁴, —O(CH₂)ₙNR³R⁴, —O(CH₂)ₙHet, —CONR³R⁴, —SO₂R⁵, —NR³CO(CH₂)ₙNR³R⁴, Het or —O(CH₂)ₙCONR³R⁴;
R² is hydrogen or methyl;
R³ and R⁴ are independently hydrogen, methyl, Het or C₁₋₄alkoxyC₁₋₄alkyl; or R³ and R⁴ together with the nitrogen atom to which they are attached form a morpholine, piperidine, pyrrolidine or piperazine ring, which ring may be further substituted by one or more substituents selected from halo —CN, —CF₃, —OH, —OCF₃, C₁₋₆alkyl and C₁₋₆alkoxy;
R⁵ is hydrogen or C₁₋₆alkyl;
Het is a 5 or 6-membered C-linked heterocyclyl group which may be saturated, unsaturated or aromatic, which may contain one or more heteroatoms selected from N, S or O and which may be substituted by C₁₋₆alkyl; and
n is 1-3.

5. A compound according to claim 1 selected from the list:
2-{4-(1-methyl-imidazol-4-yl)methyloxy]-phenyl}-4-(3-(6-methyl-pyridin-2-yl)-1H-pyrazol-4-yl)pyridine (Example 1);
2-[4-(ethylsulfonyl)phenyl]-4-[3-(6-methyl-pyridin-2-yl)-1H-pyrazol-4-yl]pyridine (Example 2);
4-[3-(6-methylpyridin-2-yl)-1H-pyrazol-4-yl]-2-[4-(pyrrolidin-1-ylmethyl)phenyl] pyridine (Example 3);
4-(4-{4-[5-methyl-3-(6-methylpyridin-2-yl)-1H-pyrazol-4-yl]pyridin-2-yl}benzyl)morpholine (Example 7); and
3-[2-(4-(2-(pyrolidin-1-yl)ethoxy)phenyl)pyridin-4-yl]-4-[6-methylpyridin-2-yl]-1H-pyrazole (Example 22);
and pharmaceutically acceptable salts, solvates and derivatives thereof.

6. A pharmaceutical composition comprising a compound defined in claim 1 and a pharmaceutically acceptable carrier or diluent.

7-10. (canceled)

11. A method for the treatment or prophylaxis of a disorder mediated by the ALK5 receptor in mammals, wherein the disorder is selected from chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis, kidney fibrosis, liver fibrosis [for example, hepatitis B virus (HBV), hepatitis C virus (HCV)], alcohol induced hepatitis, retroperitoneal fibrosis, mesenteric fibrosis, haemochromatosis and primary biliary cirrhosis, endometriosis, keloids and restenosis, which method comprises administering to a mammal in need of such treatment a compound according to claim 1.
